(19) United States
(12) Patent Application Publication
(10) Pub. No.: US 2024/0123205 A1
(43) Pub. Date: Apr. 18, 2024

(54) GEOMETRICALLY DEFORMABLE IMPLANTABLE CONTAINMENT DEVICES FOR RETENTION OF BIOLOGICAL MOIETIES

(71) Applicant: W. L. Gore & Associates, Inc., Newark, DE (US)

(72) Inventors: Edward H. Cully, Newark, DE (US); Jeffrey B. Duncan, Newark, DE (US); Erin Hutchinson, Newark, DE (US); Paul D Drumheller, Newark, DE (US); Justin Tettenborn, Newark, DE (US); Regina M Dell, Newark, DE (US)

(73) Assignee: W. L. Gore & Associates, Inc., Newark, DE (US)

(21) Appl. No.: 18/547,673

(22) PCT Filed: Feb. 25, 2021
(86) PCT No.: PCT/US21/19590

§ 371 Date: Aug. 23, 2023

(51) Int. Cl. A61M 31/00; A61K 9/00; A61K 35/12

(52) U.S. Cl. CPC A61M 31/002; A61K 9/0024; A61K 35/12; A61M 2202/09; A61M 2205/0266; A61M 2210/1042; A61M 2210/12

(57) ABSTRACT

Described herein are encapsulation devices that include an inner layer, an outer layer, a cell containment layer positioned between the inner and outer layer, and structural elements disposed within the cell containment layer to separate the inner and outer layers by a separation distance. The structural elements define reservoir spaces for the retention of a biological moiety (such as cells) therein. The structural elements maintain a separation distance both under external compressive forces and under internal expansive forces, such as undergoing a geometric change in the encapsulation device or during crushing and subsequent expansion of the cell encapsulation device. In some embodiments, a reinforcing layer is positioned between the inner layer and the cell containment layer. In addition, the encapsulation device may include at least one configuration element. Methods of placing encapsulation devices in a body conduit intra-luminally, extra-luminally, and via an open surgical procedure are also disclosed.

### FIELD

The present invention relates to the fields of implantable biological devices and biological therapies, and in particular, to encapsulation devices for housing biological moieties that are able to geometrically deform. Methods for placing an encapsulation device into and/or around a body conduit are also included.

### BACKGROUND

Biological therapies are increasingly viable methods for treating peripheral artery disease, aneurysm, heart disease, Alzheimer's and Parkinson's diseases, autism, blindness, diabetes, and other pathologies.

With respect to biological therapies in general, cells, viruses, viral vectors, bacteria, proteins, antibodies, genes, and other bioactive moieties may be introduced into a patient by surgical or interventional methods that place the bioactive moiety into a tissue bed of a patient. Often the bioactive moieties are first placed in a device that is then inserted into the patient. Alternatively, the device may be inserted into the patient first with the bioactive moiety added later.

The implantation of external devices into a body triggers an immune response in which makes it difficult, if not impossible for blood vessels to form in close proximity to the housed biological moieties, thereby restricting access to the oxygen and nutrients needed to maintain the viability and health of the encapsulated cells. When housed biological moieties are placed in or around a blood vessel, an immunological response still occurs and the cells may be crushed and/or killed during the implantation into the blood vessel. It is therefore a need in the art for containment devices that can be deployed in or around a lumen (e.g., a blood vessel) for more direct access to source of oxygen and nutrients for the housed biological moiety (e.g. cells) so that the biological moiety can survive and secrete a therapeutically useful substance.

### SUMMARY

According to one aspect, (“Aspect 1”), an implantable encapsulation device includes an inner layer, an outer layer and a containment layer positioned between the inner layer and the outer layer and including structural elements disposed therein to maintain a separation distance between the inner layer and the outer layer. The structural elements define a plurality of reservoir spaces for the placement of at least one biological moiety therein. The structural elements also maintain the separation distance both under external compressive forces and under internal expansive forces. At least one of the inner layer and the outer layer is a composite layer including a cellular open layer and a cell retentive layer. Additionally, the encapsulation device has a substantially tubular configuration and is configurable from a first tubular configuration having a first diameter to a second tubular configuration having a second diameter.

According to another aspect, (“Aspect 2”) further to Aspect 1, including a filling tube positioned between the structural elements and into at least one of the reservoir spaces for placement of the biological moiety in the reservoir spaces.

According to another aspect, (“Aspect 3”) further to Aspect 1 or Aspect 2, including a reinforcing layer positioned between the inner layer and the containment layer.

According to another aspect, (“Aspect 4”) further Aspect 1 or Aspect 2, including a reinforcing layer positioned between the outer layer and the containment layer.

According to another aspect, (“Aspect 5”) further to Aspect 1 or Aspect 2, including a reinforcing layer positioned externally to the outer layer.

According to another aspect, (“Aspect 6”) further to any one of Aspects 3 to 5, where the reinforcing layer comprises a shape memory material.

According to another aspect, (“Aspect 7”) further to any one of Aspects 1 to 6, where the inner layer is a composite layer including a cellular open layer and a first cell retentive layer and the outer layer is a second cell retentive layer.

According to another aspect, (“Aspect 8”) further any one of Aspects 1 to 7, where the outer layer is a composite layer including a cellular open layer and a first cell retentive layer and the inner layer is a second cell retentive layer.

According to another aspect, (“Aspect 9”) further to any one of Aspects 1 to 8, where the inner layer is a first composite layer comprising a first cellular open layer and a first cell retentive layer and where the outer layer is a second composite layer comprising a second cellular open layer and a second cell retentive layer.

According to another aspect, (“Aspect 10”) further to any one of Aspects 1 to 9 the structural elements comprise a shape memory material.

According to another aspect, (“Aspect 11”) further to any one of Aspects 1 to 10, where the at least one biological moiety is selected from cells, viruses, viral vectors, bacteria, proteins, antibodies, genes, DNA, RNA and combinations thereof.

According to another aspect, (“Aspect 12”) further to Aspect 11, where the cells are selected from prokaryotic cells, eukaryotic cells, mammalian cells, non-mammalian cells, stem cells and combinations thereof.

According to another aspect, (“Aspect 13”) further to any one of Aspects 1 to 12, where at least one of the inner layer the outer layer is a cellular open layer and the at least one biological moiety is microencapsulated.

According to another aspect, (“Aspect 14”) further to any one of Aspects 1 to 13, where the structural elements are adhered to at least one of the first layer and the second layer.

According to another aspect, (“Aspect 15”) further to any one of Aspects 1 to 14, where the inner layer is a first composite layer including a first cellular open layer and the outer layer is a second composite layer including a second cellular open layer, where the structural elements are adhered to the first and second cellular open layers of the first and second composite layers, and where the structural elements do not penetrate into pores of the first cellular open layer or the second cellular open layer.

According to another aspect, (“Aspect 16”) further any one of Aspects 1 to 15, where the at least two reservoir spaces that are fluidly interconnected.

According to another aspect, (“Aspect 17”) further any one of Aspects 1 to 15, where the at least two reservoir spaces are discrete.

According to one aspect (“Aspect 18”) an encapsulation device includes a first composite layer including a first cellular open layer and a first cell retentive layer, a second composite layer including a second cellular open layer and a second cell retentive layer, a containment layer positioned between the first composite layer and the second composite layer, and at least one configuration element including a shape memory material. The containment layer includes structural elements disposed therein to maintain a separation distance between the first composite layer and the second composite layer. The structural elements define a plurality of reservoir spaces for the placement of at least one biological moiety therein.

According to another aspect, (“Aspect 19”) further to Aspect 18, where the at least one configuration element is positioned between the first cellular open layer and the first cell retentive layer.

According to another aspect, (“Aspect 20”) further to Aspect 18 or Aspect 19, where the at least one configuration element is positioned between the second cellular open layer and the second cell retentive layer.

According to another aspect, (“Aspect 21”), further to any one of Aspects 18 to 20, where the at least one first configuration element is positioned between the first cellular open layer and the first cell retentive layer and at least one second configuration element is positioned between the second cellular open layer and the second cell retentive layer.

According to another aspect, (“Aspect 22”), further to any one of Aspects 18 to 20, where the at least one configuration element is exteriorly positioned on the first cellular open layer and the first cellular open layer forms an exterior surface of the encapsulation device.

According to another aspect, (“Aspect 23”), further to any one of Aspects 18 to 20, where the at least one configuration element is exteriorly positioned on the second cellular open layer and the second cellular open layer forms an exterior surface of the encapsulation device.

According to another aspect, (“Aspect 24”), further to any one of Aspects 18 to 20, where at least one configuration element replaces the structural elements and is positioned between the first composite layer and the second composite layer.

According to another aspect, (“Aspect 25”), further any one of Aspects 18 to 24, where the biological moiety is selected from cells, viruses, viral vectors, bacteria, proteins, antibodies, genes, DNA, RNA and combinations thereof.

According to another aspect, (“Aspect 26”), further to Aspect 25, where the cells are selected from prokaryotic cells, eukaryotic cells, mammalian cells, non-mammalian cells, stem cells and combinations thereof.

According to another aspect, (“Aspect 27”), further to any one of Aspects 18 to 26, where the structural elements are adhered to the first cellular open layer of the first composite layer and the second cellular open layer of the second composite layer and the structural elements do not penetrate into pores of the first cellular open layer or the second cellular open layer.

According to another aspect, (“Aspect 28”), further to any one of Aspects 18 to 27, where the plurality of reservoir spaces in the cell containment layer are interconnected.

According to another aspect, (“Aspect 29”), further to any one of Aspects 18 to 28, where the plurality of reservoir spaces in the cell containment layer are discrete.

According to another aspect, (“Aspect 30”), further to any one of Aspects 18 to 29, where the structural elements maintain the separation distance both under external compressive forces and under internal expansive forces.

According to one aspect, (“Aspect 31”) a method of placing an intra-luminal device includes accessing a body conduit that has an inner surface, tracking a catheter to a target location point on the body conduit, and deploying an encapsulation device within the body conduit, where the encapsulation device is configured to fit the body conduit. The encapsulation device includes at least one reservoir space containing a biological moiety therein, a first cellular open layer on a first side of the at least one reservoir space where the first cellular open layer faces the inner surface of the body conduit, and a first cell retentive layer on a second side of the reservoir space.

According to another aspect, (“Aspect 32”), further to Aspect 31, where the encapsulation device includes a second cellular open layer adjacent to the first cell retentive layer where the second cellular open layer faces a center portion of the body conduit.

According to another aspect, (“Aspect 33”), further to Aspect 32, where the encapsulation device includes a second cell retentive layer positioned between the first cellular open layer and the at least one reservoir space.

According to another aspect, (“Aspect 34”), further to any one of Aspects 31 to 33, where the at least one reservoir space is positioned within a containment layer containing structural elements therein which define the at least one reservoir space.

According to another aspect, (“Aspect 35”), further to Aspect 34, where the structural elements maintain a separation distance both under external compressive forces and under internal expansive forces.

According to another aspect, (“Aspect 36”), further to any one of Aspects 31 to 35 including inserting a guidewire and tracking a catheter over a guidewire to the target location point.

According to another aspect, (“Aspect 37”), further to any one of Aspects 31 to 36 where the encapsulation device is constrained within the catheter.

According to another aspect, (“Aspect 38”), further to any one of Aspects 31 to 37 where the encapsulation device is endoscopically deployed, cystoscopically deployed, laparoscopically deployed, or bronchoscopically deployed.

According to another aspect, (“Aspect 39”), further to any one of Aspects 31 to 38 where the biological moiety releases a therapeutic product to the body conduit.

According to another aspect, (“Aspect 40”), further to any one of Aspects 31 to 39 where the body conduit is a blood vessel.

According to another aspect, (“Aspect 41”), further to any one of Aspects 31 to 39 where the body conduit is a gastrointestinal conduit.

According to an Aspect, (“Aspect 42”) a method of placing an intra-luminal device includes accessing a body conduit at a target location point, tracking a catheter to the target location point, and deploying an encapsulation device at the target location point. The encapsulation device includes at least one reservoir space containing therein a biological moiety. The at least one reservoir space is positioned within a containment layer that has at least one structural element located therein. A separation distance is maintained during deployment of the encapsulation device.

According to another aspect, (“Aspect 43”), further to Aspect 42 including inserting a guidewire and tracking a catheter over a guidewire to the target location point.

According to another aspect, (“Aspect 44”), further to Aspect 43 where the encapsulation device is constrained within the catheter.

According to another aspect, (“Aspect 45”), further to any one of Aspects 42 to 44 where the encapsulation device is endoscopically deployed, cystoscopically deployed, laparoscopically deployed, or bronchoscopically deployed.

According to another aspect, (“Aspect 46”), further to any one of Aspects 42 to 45 where the biological moiety releases a therapeutic product to the body conduit.

According to another aspect, (“Aspect 47”), further to any one of Aspects 42 to 46 where the body conduit is a blood vessel.

According to another aspect, (“Aspect 48”), further to any one of Aspects 42 to 46 where the body conduit is a gastrointestinal conduit.

According to another aspect, (“Aspect 49”), further to any one of Aspects 42 to 48 including a cellular open layer positioned adjacent to an inner surface of the body conduit.

According to another aspect, (“Aspect 50”), further to any one of Aspects 42 to 49 including a cell retentive layer as an inner layer of the encapsulation device.

According to another aspect, (“Aspect 51”), further to any one of Aspects 42 to 50 where a first cellular open layer is positioned on a first side of the at containment layer and a second cellular open layer is positioned on a second side of the containment layer.

According to one aspect, (“Aspect 52”) a method of placing an extra-luminal device includes accessing a first body conduit at a first target entry point, inserting a guidewire into the first body conduit at the first target entry point, tracking a catheter over the guidewire to a target exit point on the first body conduit, using an accessory tool to exit the first body conduit at a target exit point, tracking the guidewire to a second target entry point on a second body conduit, using the accessory tool to enter the second body conduit, deploying an encapsulation device at the second target entry point on the second body conduit. The encapsulation device includes at least one reservoir space containing a biological moiety therein where the at least one reservoir space has a first cell retentive layer thereon. The encapsulation device forms a third conduit connecting the first body conduit and the second body conduit. And the cell retentive layer forms an inner layer of the third body conduit.

According to another aspect, (“Aspect 53”), further to Aspect 52 where the encapsulation device is constrained within the catheter.

According to another aspect, (“Aspect 54”), further to Aspect 51 or Aspect 53 where the encapsulation device is endoscopically deployed, cystoscopically deployed, laparoscopically deployed, or bronchoscopically deployed.

According to another aspect, (“Aspect 55”), further to any one of Aspects 52 to 54 where the biological moiety releases a therapeutic product.

According to another aspect, (“Aspect 56”), further to any one of Aspects 52 to 55 where the body conduit is a blood vessel.

According to another aspect, (“Aspect 57”), further to any one of Aspects 52 to 55 where the body conduit is a gastrointestinal conduit.

According to another aspect, (“Aspect 58”), further to any one of Aspects 52 to 57 where the encapsulation device includes a containment layer that includes the at least one reservoir space and structural elements that maintain a separation distance under external compressive forces and under internal expansive forces.

According to another aspect, (“Aspect 59”), further to any one of Aspects 52 to 58 where the encapsulation device includes a second cell retentive layer on a side of the at least one reservoir space opposing the first cell retentive layer.

According to one aspect, (“Aspect 60”) a method of placing an extra-luminal device includes accessing a body conduit at a first target entry point, inserting a guidewire into the body conduit at the first target entry point, tracking a catheter over the guidewire to a target exit point located before a desired bypassed region of the body conduit, tracking a catheter over the guidewire to a target exit point located before a desired bypassed region of the body conduit, using an accessory tool to exit the body conduit at the first target exit point, tracking the guidewire to a second target entry point on the body conduit at a location after the desired bypassed region of the body conduit, and deploying an encapsulation device at the second target entry point such that the encapsulation device connects the first target exit point and the second target entry point. The encapsulation device incudes at least one reservoir space containing a biological moiety therein and a cellular open layer. A portion of the cellular open layer is adjacent to an inner surface of the body conduit.

According to another aspect, (“Aspect 61”), further to Aspect 60 where the encapsulation device is constrained within the catheter.

According to another aspect, (“Aspect 62”), further to Aspect 60 or Aspect 61 where the encapsulation device is endoscopically deployed, cystoscopically deployed, laparoscopically deployed, or bronchoscopically deployed.

According to another aspect, (“Aspect 63”), further to any one of Aspects 60 to 62 where the biological moiety releases a therapeutic product into the body conduit.

According to another aspect, (“Aspect 64”), further to any one of Aspects 60 to 63 where the body conduit is a blood vessel.

According to another aspect, (“Aspect 65”), further to any one of Aspects 60 to 63 where the body conduit is a gastrointestinal conduit.

According to another aspect, (“Aspect 66”), further to any one of Aspects 60 to 65 where the desired bypassed region is an occlusion.

According to one aspect (“Aspect 67”) a method of placing an encapsulation device includes surgically accessing a target location point on a first body conduit at a first target entry point, resecting a portion of the body conduit at the target location point, and replacing the resected portion of the body conduit at the target location point with an encapsulation device via end-to-end anastomosis. The encapsulation device includes structural elements forming reservoir spaces in a containment layer and the reservoir spaces contain therein a biological moiety.

According to another aspect, (“Aspect 68”) further to Aspect 67 where the containment layer has a first side and where a cell retentive layer is positioned on the first side of the containment layer and a cellular open layer is positioned on the cell retentive layer and is adjacent to an inner surface of the body conduit.

According to another aspect, (“Aspect 69”) further to Aspect 67 or Aspect 68 where the biological moiety releases a therapeutic product into the body conduit.

According to another aspect, (“Aspect 70”) further to any one of Aspects 67 to 69 where the body conduit is a blood vessel.

According to another aspect, (“Aspect 71”) further to any one of Aspects 67 to 70 the body conduit is a gastrointestinal conduit.

According to one aspect, (“Aspect 72”) a method of placing an encapsulation device includes surgically accessing a target location point on a body conduit, forming a slit in the body conduit at the target location point to access the body conduit, and inserting an encapsulation device into the body conduit at the target location point. The encapsulation device includes at least one reservoir space containing a biological moiety therein and the at least one reservoir space has a cellular open layer thereon which is positioned adjacent an inner surface of the body conduit.

According to another aspect, (“Aspect 73”) further to Aspect 72 where the biological moiety releases a therapeutic product into the body conduit.

According to another aspect, (“Aspect 74”) further to Aspect 72 or Aspect 73 where the body conduit is a blood vessel.

According to another aspect, (“Aspect 75”) further to Aspect 72 or Aspect 73 where the body conduit is a gastrointestinal conduit.

According to one aspect, (“Aspect 76”) a method of placing an extra-luminal bypass or shunt device includes surgically accessing a first target location point on a first body conduit, connecting a first end of an encapsulation device to the first target location point of the first body conduit, and connecting a second end of the encapsulation device to a second target location point on a second body conduit. The encapsulation device includes a cell retentive layer and at least one reservoir space containing therein at least one biological moiety. The encapsulation device forms a third conduit connecting the first body conduit and the second body conduit. The cell retentive layer forms an interior surface of the third conduit.

According to another aspect, (“Aspect 77”) further to Aspect 76 where the at least one biological moiety releases a therapeutic product.

According to another aspect, (“Aspect 78”) further to Aspect 76 or Aspect 77, the first body conduit and second body conduit are each blood vessels.

According to another aspect, (“Aspect 79”) further to Aspect 76 or Aspect 77, where the first body conduit and second body conduit are each a gastrointestinal conduit.

According to one aspect, (“Aspect 80”) a method of placing an encapsulation device includes surgically accessing a target location point of a body conduit and placing an encapsulation device around an external surface of the body conduit such that the encapsulation device substantially surrounds at least a portion of the body conduit. The encapsulation device includes at least one reservoir space containing therein a biological moiety. The at least one reservoir space has a cellular open layer positioned on a side thereof and the cellular open layer is adjacent to the outside surface of the body conduit.

According to another aspect, (“Aspect 81”) further to Aspect 80 where the at least one biological moiety releases a therapeutic product.

According to another aspect, (“Aspect 82”) further to Aspect 80 or Aspect 81, the first body conduit and second body conduit are each blood vessels.

According to another aspect, (“Aspect 83”) further to Aspect 80 or Aspect 81, the first body conduit and second body conduit are each a gastrointestinal conduit.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a schematic illustration of a cross-section of a tubular encapsulation device having a composite inner layer and a composite outer layer according to embodiments described herein;

FIG. 1B is a schematic illustration of the a cross-section of the tubular encapsulation device of 1A having containing therein a reinforcing layer according to embodiments described herein;

FIG. 2 is a schematic illustration of a cross-section of a tubular encapsulation device having a composite inner layer and an outer layer containing only a cell retentive layer according to embodiments described herein;

FIG. 3 is a schematic illustration of a cross-section of a tubular encapsulation device having a composite outer layer and an inner layer containing only a cell retentive layer according to embodiments described herein;

FIG. 4 is schematic illustration of a cross-section of a porous material used to construct encapsulation devices in accordance with embodiments described herein;

FIGS. 5A-5C are schematic illustrations of cross-sections of encapsulation devices containing configuration elements to enable a geometric shape change according to embodiments described herein;

FIG. 6 is a schematic illustration of a cross-section of a portion of an encapsulation device that is configurable from a first geometric shape to a second geometric shape according to embodiments described herein;

FIGS. 7A-7B are planar frames formed of a shape memory material according to embodiments described herein; and

FIGS. 7C-7D are illustrations of the non-planar form of FIGS. 7A and 7B, respectively, according to embodiments described herein.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting. It is to be appreciated that the terms “encapsulation device” and “housing device” may be used interchangeably herein.

Described herein are devices for housing biological moieties, where the biological moieties are implanted intravascularly or extra-vascularly into or around a conduit (e.g., blood vessel) of a patient to provide biological therapy. Biological moieties suitable for encapsulation and implantation using the devices described herein include cells, viruses, viral vectors, bacteria, proteins, antibodies, genes, DNA, RNA, and other bioactive moieties. In some embodiments herein, the biological moiety is a cell, but nothing in this description limits the biological moiety to cells or to any particular type of cell, and the following description applies also to biological moieties that are not cells.

Various types of prokaryotic and eukaryotic cells, mammalian cells, non-mammalian cells, and stem cells may be used with the device for encapsulating biological moieties, also referred to herein as cell containment device. In some embodiments, the cells secrete a therapeutically useful substance. Such therapeutically useful substances include hormones, growth factors, trophic factors, neurotransmitters, lymphokines, antibodies, or other cell products which provide a therapeutic benefit to the device recipient. Examples of such therapeutic cell products include, but are not limited to, hormones, growth factors, trophic factors, neurotransmitters, lymphokines, antibodies or other cell products which provide a therapeutic benefit to the device recipient. Examples of such therapeutic cell products include, but are not limited to, insulin, growth factors, interleukins, parathyroid hormone, erythropoietin, transferrin, collagen, elastin, tropoelastin, exosomes, vesicles, genetic fragments, and Factor VIII. Non-limiting examples of suitable growth factors include vascular endothelial growth factor, platelet-derived growth factor, platelet-activating factor, transforming growth factors bone morphogenetic protein, activin, inhibin, fibroblast growth factors, granulocyte-colony stimulating factor, granulocyte-macrophage colony stimulating factor, glial cell line-derived neurotrophic factor, growth differentiation factor-9, epidermal growth factor, and combinations thereof.

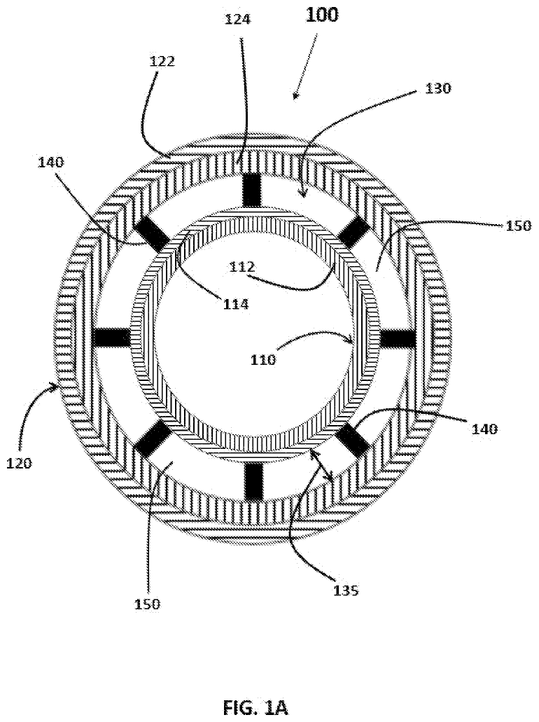

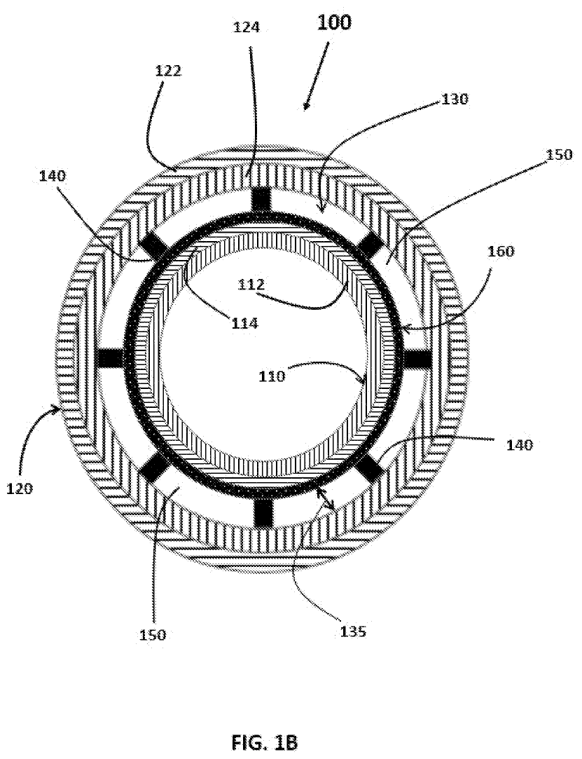

FIG. 1A is a schematic illustration of a cross-section of a tubular encapsulation device 100 that includes an inner layer 110, an outer layer 120, and at least one containment layer 130 positioned between the inner and outer layers 110, 120. The containment layer 130 includes structural elements 140 that maintain a separation distance 135 during a geometric change of the device 100. The structural elements 140 maintain a separation distance 135 from a first diameter to a second diameter (e.g., during crushing and subsequent expansion of the device 100). Additionally the separation distance 135 is maintained both under external compressive forces and under internal expansive forces. The structural elements 140 define a plurality of reservoir spaces 150 for the placement of a biological moiety within the containment layer 130. The separation distance 135 may be up to 100 microns. In some embodiments, the separation distance 135 may be at least about 100 microns, at least about 150 microns, at least about 200 microns, at least about 250 microns, or at least about 500 microns (or more). In some embodiments, the separation distance 135 may range from about 150 microns to about 500 microns, from about 200 microns to about 500 microns, from about 100 microns to about 250 microns, or from about 150 microns to about 250 microns. In one embodiment, maintaining the separation distance 135 may place the inner layer 110 in a substantially parallel relationship with the outer layer 120.

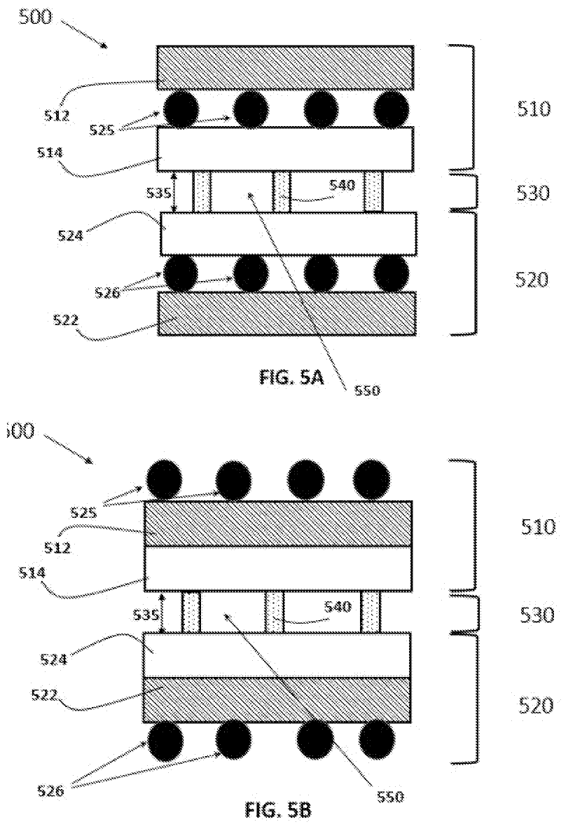

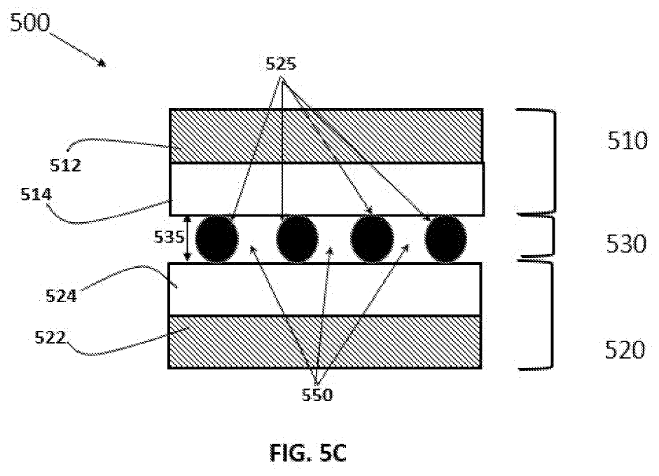

In some embodiments, such as is generally depicted in FIG. 1B, a reinforcing layer 160 may be positioned between the inner layer 110 and the containment layer 130. In other embodiments, the reinforcing layer 160 may be positioned between the outer layer 120 and the containment layer 130. In further embodiments, the reinforcing layer 160 may be externally positioned (e.g., external to the outer layer 120 or the inner layer 110). In some embodiments, the structural elements 140 are themselves reinforcing and may be formed of a shape memory material, such as, for example, configuration elements 525, 526 illustrated in FIGS. 5A-5C and which are described in detail below. It is to be appreciated that the open layer 110 and/or the outer layer 120 may contain a single layer or be formed of a composite layer as described herein. Non limiting examples of suitable materials for the reinforcing layer 160 include a metallic element (e.g., a metal stent), a shape memory material, a porous material, or a non-porous material as described herein. In some embodiments, the cross-section of the encapsulation device 100 may have a generally cylindrical, ovoid, or elliptical shape.

In some embodiments, at least one of the inner and outer layers 110, 120 includes a composite layer. FIGS. 1A and 1B depict embodiments where the inner layer 110 and the outer layer 120 are composite layers. The inner layer 110 includes a cellular open layer 112 and a cell retentive layer 114 disposed adjacent to the cellular open layer 112. The outer layer 120 may also be a composite layer that includes a cellular open layer 122 disposed adjacent to a cell retentive layer 124. A cell retentive layer 114, 124 is positioned on either side of the containment layer 130 such that the containment layer 130 (and reservoir spaces 150 therein) are sandwiched between the cell retentive layers 114, 124. The cellular open layers 112, 122 of the inner and outer layers 110, 120 may include or be formed of the same material or different materials. Likewise, the cell retentive layers 122, 124 of the inner and outer layers 110, 120 may include or be formed of the same material or different materials. Additionally, the cellular open layers 112, 122 may have porosities that are less than the porosities of the cell retentive layers. It is to be appreciated that embodiments where the porosities of the cellular open layers 112, 122 have porosities that are equal to, or larger than, the porosities of the cell retentive layers are considered within the purview of this disclosure. In the embodiments depicted in FIGS. 1A and 1B, host tissue ingrowth such as host vascular tissue, may occur into both the cellular open layer 112 and the cellular open layer 122. The cellular open layers 112, 122 are sufficiently porous to permit growth of a tissue, such as vascular tissue, from a patient into the pores of the cellular open layers 112, 122 up to, but not through, the cell retentive layers 114, 124.

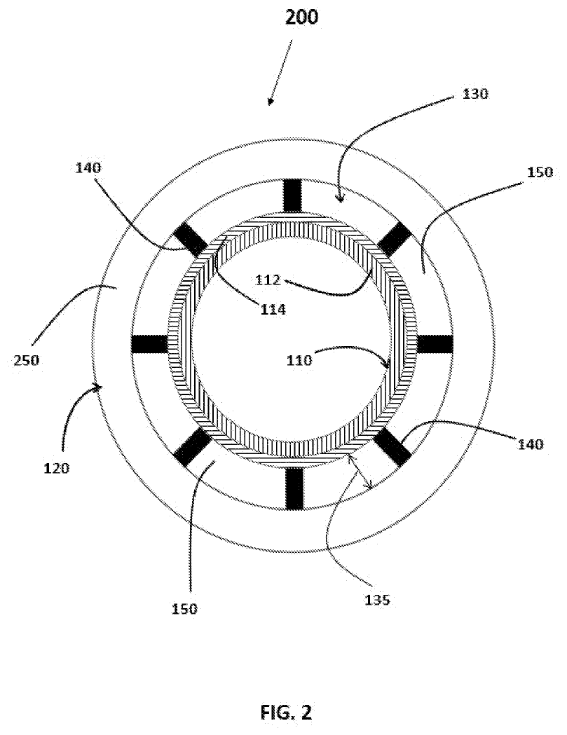

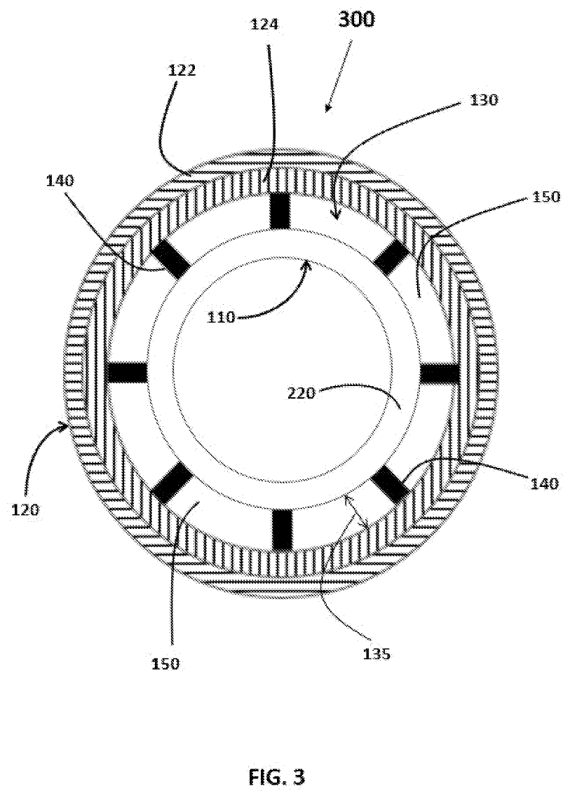

In some embodiments, only one of the inner and outer layers of the encapsulation device is a composite layer. In at least one embodiment, such as is shown in FIG. 2, the first layer 110 of the encapsulation device 200 may be a composite layer that includes a cellular open layer 112 and a cell retentive layer 114, while the outer layer 210 includes only a cell retentive layer 250. A containment layer 130 may be positioned between the first layer 110 and the cell retentive layer 250, and more specifically, between the cell retentive layer 114 and cell retentive layer 250. The containment layer 130 contains structural elements 140 that define a plurality of reservoir spaces 150 for the placement of one or more biological moiety. In another embodiment, such as is depicted in FIG. 3, the outer layer 120 of the encapsulation device 300 may be a composite layer that includes a cellular open layer 122 and a cell retentive layer 124, while the inner layer 110 may only include only a cell retentive layer 220 A containment layer 130 containing therein reservoir spaces 150 may be positioned between the cell retentive layer 220 and the outer layer 120. More specifically, the containment layer 130 may be positioned between the cell retentive layer 114 and cell retentive layer 220. It is to be appreciated that the cellular open layers 112, 122 allow ingrowth of host tissue cells. As with the embodiments described above, cell retentive layers 114, 250 (FIG. 2) and cell retentive layers 220, 124 (FIG. 3) are located on both sides of the containment layer 130 in which the structural elements 140 and reservoir spaces 150 (and biological moieties) are contained. Also, the structural elements 140 define a separation distance 135 that is maintained both under external compressive forces and under internal expansive forces. It is to be appreciated that the embodiments depicted in FIGS. 1A, 1B, 2, and 3 are exemplary in nature, and that there may be various orientations of the cellular open layers, the cell retentive layers, and the reinforcement element(s) 160 and are considered to be within the purview of this disclosure.

In some embodiments, the cell retentive layers are impervious to cell ingrowth. For example, in some embodiments, both cell retentive layers have an average pore size that is sufficiently small so as to prevent host tissue ingrowth, such as vascular tissue ingrowth. As one non-limiting embodiment, the average pore size of the cell retentive layers may be less than about 5 microns, less than about 1 micron, or less than about 0.5 microns, as measured by porometry. A small pore size allows the cell retentive layers to keep the biological moiety disposed in the reservoir spaces of the containment layer inside the encapsulation device.

At least encapsulation devices 100, 200, 300 described herein are configurable from a first tubular configuration having a first diameter to a second tubular configuration having a second diameter, such as, for example, when the device is physically crushed and placed into a catheter for insertion into a patient. In at least one embodiment, the encapsulation device is deployed using a minimally invasive procedure. The delivery system most commonly includes a restraining member that maintains a device in a second tubular configuration (i.e., collapsed or crushed state) for delivery through a body conduit (e.g. a blood vessel) to a desired site. Once the device is positioned at the desired site, the restraining member is released so that the encapsulation device may expand or be expanded to its first tubular configuration (i.e., expanded state). The structural elements described herein maintain the separation distance during the crushing, delivery, and subsequent expansion back to the original shape and size (e.g., shape prior to crushing).

Various cell types can grow into the cellular open layer(s) of an encapsulation device as described herein. The predominant cell type that grows into a particular porous material (e.g., a cellular open layer) depends primarily on the implantation site, the composition, and permeability of the material, and any biological factors that may be incorporated in the material or introduced through porous material(s). In some embodiments, vascular tissue is the predominant cell type that grows into the cellular open layer(s) of the encapsulation device. In other embodiments, hepatic tissue is the predominant cell type that grows into a porous material for use in the encapsulation device. In further embodiments, osseous tissue is the predominant cell type that grows into a porous material for use in the encapsulation device. In other non-limiting embodiments, gastrointestinal tissue, urinary tract tissue, respiratory tissue, neurological tissue, lymphatic tissue, and connective tissue is the predominant cell type that grows into the porous layer(s) (e.g., cellular open layer(s)) in the encapsulation device. Vascularization of the cellular open layer by a well-established population of vascular cells in the form of a capillary network is encouraged to occur as a result of neovascularization of the material from tissues of a patient into and across the thickness of the cellular open layer next to the interior surface of the device, but not across the cell retentive layer.

In a further embodiment, neither the first nor the second layers is a composite layer, but rather only includes a cell retentive layer. In an embodiment where the encapsulation device includes only cell retentive layers and no cellular open layer(s), the encapsulation device optionally could be used with a housing that is, or can be, disposed in a patient, and that is made from a material that allows engraftment of surrounding tissue. In some embodiments, the housing may be implanted into a patient for a period of time sufficient to allow engraftment of surrounding tissue before the device is inserted into the housing. In other embodiments, the device and the housing may be inserted into a patient together.

In yet another embodiment, neither the first nor the second layer is a composite layer. Instead, the first and/or second layers are cellular open layers which permit some degree of host cell penetration into the encapsulation device, or which permit host cell penetration and vascularization into the encapsulation device. In addition, the biological moiety within the containment layer are free to migrate in and out of the encapsulation device. In some embodiments, the biological moiety to be inserted into the encapsulation device may be microencapsulated within a biomaterial of natural or synthetic origin, including, but not limited to, a hydrogel biomaterial. The encapsulation provides isolation for the biological moiety (e.g. cells) from host immune response.

Materials useful as the first and second porous layers present in a composite layer include, but are not limited to, alginate, cellulose acetate, polyalkylene glycols such as polyethylene glycol and polypropylene glycol, panvinyl polymers such as polyvinyl alcohol, chitosan, polyacrylates such as polyhydroxyethylmethacrylate, agarose, hydrolyzed polyacrylonitrile, polyacrylonitrile copolymers, polyvinyl acrylates such as polyethylene-co-acrylic acid, porous polytetrafluoroethylene (PTFE), modified polytetrafluoroethylene polymers, tetrafluoroethylene (TFE) copolymers, porous polyalkylenes such as porous polypropylene and porous polyethylene, porous polyvinylidene fluoride, porous polyester sulfone (PES), porous polyurethanes, porous polyesters, and copolymers and combinations thereof. In some embodiments, the materials useful as an outer porous layer include biomaterial textiles.

In some embodiments, one or both of the inner layer and the outer layer of the encapsulation device is made, primarily or entirely, of a porous material having selective sieving and/or porous properties. The porous material may be manufactured from any biologically compatible material having the appropriate permeability characteristics. The porous material controls the passage of solutes, biochemical substances, viruses, and cells, for example, through the material, primarily on the basis of size. Non-limiting examples of suitable porous materials include, but are not limited to, one or more of the materials set forth above for the inner and outer layers, including biomaterial textiles.

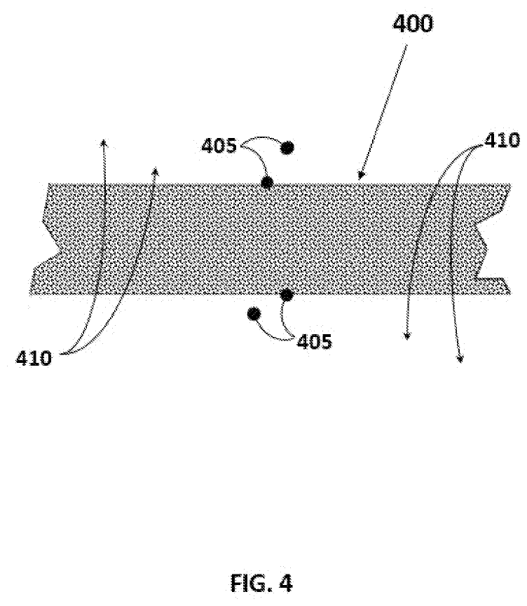

In embodiments where the porous material is porous only through a portion of its thickness, the molecular weight cutoff, or sieving property, of the porous membrane begins at the surface. As a result, certain solutes and/or bioactive moieties such as cells do not enter and pass through the porous spaces of the material from one side to the other. FIG. 4 depicts a cross-sectional view of a porous material 400 useful in encapsulation devices described herein, where the selective permeability of the porous material 400 excludes cells 405 from migrating or growing into the spaces of the porous material 400 while permitting bi-directional flux of solutes 410 across the thickness of the porous material 400. Vascular endothelial cells can combine to form capillaries thereon. Additional cells types can combine to form an organized or disorganized tissue thereon, such as, hepatic tissue cells, neurological tissue cells, lymphatic tissue cells, gastrointestinal tissue cells, connective tissue cells urinary tract tissue cells, respiratory tissue cells, and osseous tissue cells. Such capillary formation or neovascularization or organized or disorganized tissue formation of the porous material 400 permits fluid and solute flux between tissues of a patient and the contents of the encapsulation device to be enhanced.

In some embodiments, the inner and outer layers are flexible, but the structural elements maintain the encapsulation device as a generally tubular structure in its expanded configuration. The structural elements maintain a separation distance under an applied force over the diameter of the device. The applied force may be an external compressive force that would tend to cause the reservoir space(s) between the first and second layers to collapse in the absence of the structural elements. For example, a clinician may exert a compressive force to crush the tubular device prior to or during insertion. If the external compressive force decreases the distance between the inner layer and the outer layer of the encapsulation device, cells (i.e., biological moieties) within the encapsulation device may be subjected to undesirable mechanical stimuli such as a compressive stimuli which could result in minimized cell functionality or cell fatality.

Alternatively, the applied force may be an internal expansive force that would tend to cause the containment layer between the inner and outer layers to expand to a rounded, balloon-like membrane in the absence of the structural elements. For example, pressure may be required to inject a biological moiety (e.g. a plurality of cells) into the reservoir spaces. In one example, pressure can be caused by over-inflation at the time of insertion, e.g., due to operator error. In another example, pressure can be caused by an increase of cells due to cellular propagation and multiplication (e.g. pillowing). It is to be appreciated that the structural elements maintain the separation distance both under external compressive forces and under internal expansive forces.

The structural elements divide the containment layer into at least two reservoir spaces. Boundaries of the reservoir spaces are defined by the structural elements, the outer layer, and inner layer (or reinforcement layer if present). The number of reservoir spaces is not particularly limited and the cell containment layer may contain up to 100,000 or more reservoir spaces. In some embodiments, at least two reservoir spaces are interconnected by channels formed by and among the structural elements. In other embodiments, at least two of the reservoir spaces are discrete (i.e., are not fluidly connected). In other embodiments, a portion of the reservoir space may be interconnected and another portion of the reservoir space may be discrete (not connected).

In some embodiments, one or both of the inner and outer layers and/or the containment layer is or includes a bio-absorbable material. The bio-absorbable material may be formed as a solid (molded, extruded, or crystals), a self-cohered web, a raised webbing, or a screen. In some embodiments, one or more layers of bio-absorbable material are attached to a non bio-absorbable material having macroscopic porosity to allow for cell permeation (e.g., a cellular openlayer) to form a composite. In other embodiments, a non bio-absorbable material having microscopic porosity to decrease or prevent cell permeation is releasably attached to the porous self-cohered web to permit atraumatic removal of the encapsulation device from the body of a patient days following implantation. Resorbing into the body can promote favorable type 1 collagen deposition, tissue integration, neovascularization, and a reduction of infection. Certain materials, such as, for example, perfluorocarbon emulsions, fluorohydrogels, silicone oils, silicone hydrogels, soybean oils, silicone rubbers, and polyvinyl chloride and combinations thereof are known to have high oxygen solubility. Such highly oxygen permeable materials provide enhanced transport of oxygen into the encapsulation device from the host tissue. Such materials can be utilized as the structural elements, or may be applied, for example, as a coating or a filler onto the structural elements.

FIGS. 5A-5C are schematic illustrations of cross-sections of encapsulation devices 500 that include a first composite layer 510, a second composite layer 520, a containment layer 530 positioned between the first and second composite layers 510, 520, and at least one structural element 540 disposed within the containment layer 530 to separate the first and second composite layers 510, 520 by a separation distance 535. The containment layer 530 includes the structural elements 540 such that the separation distance 535 is maintained during a geometric change of the encapsulation device. Also, the structural elements 540 define a separation distance 535 that is maintained both under external compressive forces and under internal expansive forces. The separation distance 535 between the first and second composite layers is up to 100 microns. In some examples the separation distance between the first and second composite layers is at least 100 microns, e.g., at least 150 microns or at least 200 microns. In some examples, the separation distance may be about 250 microns, at least 250 microns, or 500 microns or more. In some embodiments, the separation distance 535 may range from about 150 microns to about 500 microns, from about 200 microns to about 500 microns, from about 250 microns to about 500 microns, from about 100 microns to about 250 microns, or from about 150 microns to about 250 microns. The structural elements 540 define a plurality of reservoir spaces 550 for the placement of a biologic moiety (not shown) within the containment layer 530. The cellular open layers 512, 524 may include or be formed of the same material or different materials. Likewise, the cell retentive layers 512, 522 may include or be formed of the same material or different materials. The cellular open layers 512 and 522 may have porosities that are less than the porosities of the cell retentive layers 514 and 524.

Turning to FIG. 5A, in some embodiments, the first composite layer 510 includes at least one first configuration element 525 positioned between the first cellular open layer 512 and the first cell retentive layer 514. The second composite layer 520 also includes at least one second configuration element 526 positioned between the second cellular open layer 522 and the second cell retentive layer 524. The first and second configuration elements 525, 526 may be formed of the same material or different materials. Non-limiting examples of materials utilized in the first and/or second configuration elements 525, 526 include a shape changing material, including, but not limited to shape memory alloys, such as nitinol, and shape memory polymers such as polyetheretherketone, polymethyl methacrylate, polyethyl methacrylate, polyacrylate, poly-alpha-hydroxy acids, polycaprolactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, polyurethanes, and copolymers or polymer blends thereof. In at least one embodiment, one or more of the configuration elements is a nitinol stent. The configuration elements induce the device into a geometric change, such as, for example, from a first geometry (e.g., a generally planar configuration) to a second geometry (e.g., a generally cylindrical configuration). The encapsulation device s are also configurable from a planar configuration to a non-planar configuration, e.g., a folded or a “jelly roll” configuration, a hyperbolic (e.g., a hot dog bun), or a furled cylindrical configuration. The configuration elements 525, 526 also facilitate implantation, including facilitating any change in profile of the encapsulation device during implantation. It is to be appreciated that one or more of the configuration elements 525, 526 may be a reinforcing layer 160 as described above with reference to FIGS. 1-3.

The cellular open layers 512, 522 may include or be formed of the same material or different materials. Likewise, the cell retentive layers 514, 524 may include or be formed of the same material or different materials. The cellular open layers 512, 522 may have porosities that are less than the porosities of the cell retentive layers 514 and 524.

In another embodiment, as shown in FIG. 5B, at least one configuration element 525 is positioned exteriorly on the encapsulation device 500. In particular, at least one first configuration element 525 is positioned at the outermost layer and is adjacent to the cellular open layer 512. Additionally at least one second configuration element 526 is positioned exteriorly on the cellular open layer 522. In particular, at least one second configuration element 526 is positioned adjacent to the cellular open layer 522 and forms an outer layer of the encapsulation device 500. FIG. 5C depicts an embodiment where at least one configuration element 525 is positioned between the cell retentive layers 514, 524 and replaces the structural elements. It is to be appreciated that FIGS. 5A-5C are exemplary in nature, and that there may be various orientations of the cellular open layers, the cell retentive layers, and the configuration element(s) 525, 526 and are considered to be within the purview of this disclosure.

In some embodiments, the first and second composite layers 510, 520 are flexible. The structural elements 540 separate the first and second composite layers 510, 520 such that there is a separation distance 535 between the composite layers 510 520. The structural elements 540 maintain that separation distance 535 under an applied force. As discussed above, the applied force may be an external compressive force that would tend to cause the encapsulation layer 530 between the first and second composite layers 510, 520 to collapse in the absence of the structural elements 540. Alternatively, the applied force may be an internal expansive force that would tend to cause the encapsulation layer between the first and second composite layers to expand to a rounded, balloon-like membrane in the absence of the structural elements. The structural elements 540 maintain the separation distance 535 both under external compressive forces and under internal expansive forces.

Similar to that which is described above, the structural elements 540 divide the containment layer 530 into at least two reservoir spaces 550. Boundaries of the reservoir spaces 550 are defined by the structural elements 540, the first composite layer 510, and the second composite layer 520. In FIGS. 5A and 5B, the reservoir spaces 550 are defined by the structural elements 540, the first cell retentive layer 514, and the second cell retentive layer 524. The number of reservoir spaces 550 is not particularly limited and the containment layer 530 may contain up to 100,000 or more reservoir spaces 550. In some embodiments, at least two reservoir spaces 550 are interconnected by channels formed by and among the structural supports 540. In other embodiments, the reservoir spaces 550 may be discrete (i.e., are not fluidly connected). In other embodiments, a portion of the reservoir space 550 may be interconnected and another portion of the reservoir space 550 may be discrete (not connected).

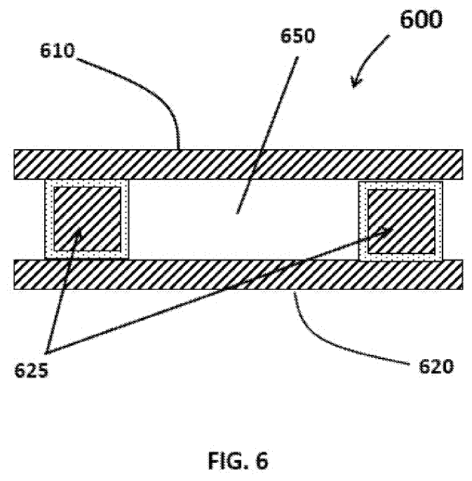

In another embodiment depicted in FIG. 6, an encapsulation device 600 is depicted that includes a first porous layer 610, a second porous layer 620, and at least one configuration element 625. At least one reservoir space 650 is formed by the boundaries of the first porous layer 610, the second porous layer 620, and the configuration elements 625. In some embodiments, the configuration elements 625 include or is formed of a shape memory material as described above such that the encapsulation device 600 is configured to geometrically change from a planar configuration to a non-planar configuration. In some embodiments, the geometric change may be from a non-planar configuration to a planar configuration.

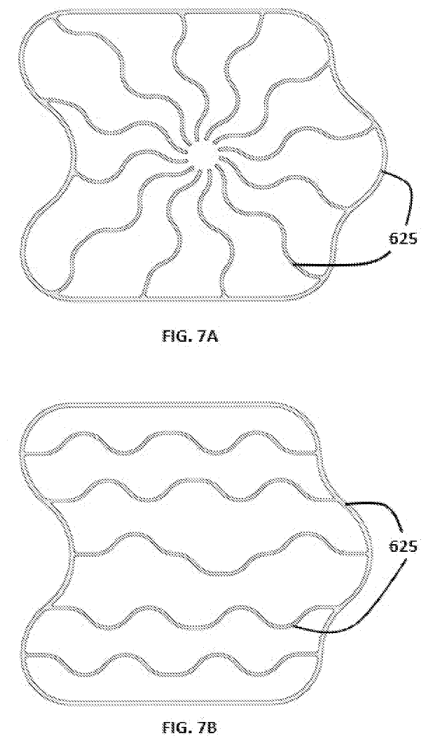

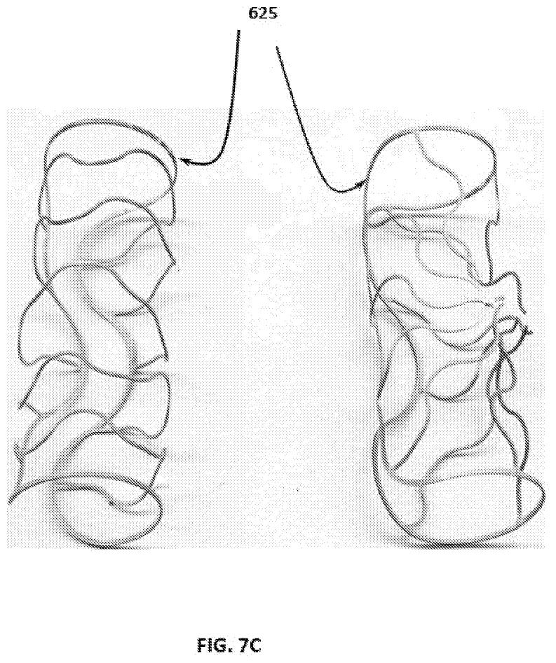

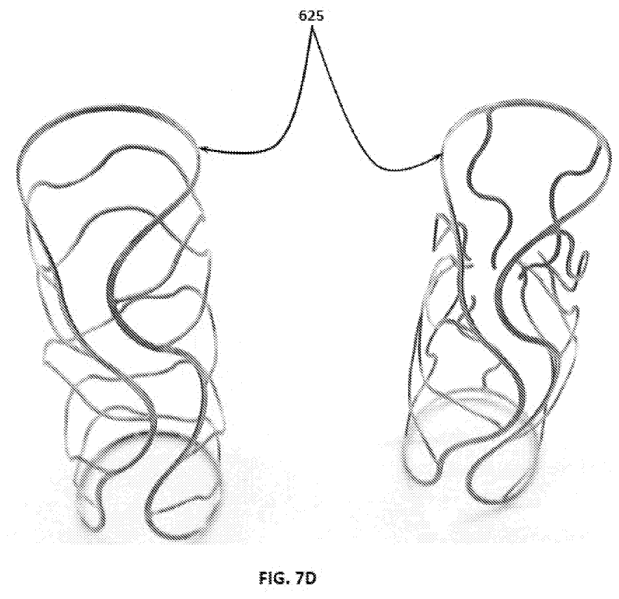

In some embodiments, the configuration element 625 is in the form of a frame formed of a shape memory material. Non-limiting examples of suitable frames for use in the encapsulation devices described here include those depicted in FIGS. 7A and 7B. It is to be appreciated that FIGS. 7A- and 7B are exemplary in nature and in no way constrict this disclosure to these particular designs. The configuration element 625 may have a planar configuration, such as is depicted in FIGS. 7A and 7B. After implantation, the configuration element 625 of FIGS. 7A and 7B take a non-planar configuration, such as is depicted in FIGS. 7C and 7D, respectively. In use, a cellular open layer and a cell retentive layer (not depicted) are non-removably positioned on either side of the configuration element 625. After implanted in a patient, the encapsulation device undergoes a geometric change from a first geometric configuration (e.g., a planar configuration) to a second geometric configuration (a circular configuration). In some embodiments, the encapsulation may undergo a geometric change from a planar configuration to, for example, a circular configuration, a jelly roll configuration, a hyperbolic configuration, a furled configuration, or a folded configuration.

The encapsulation devices described herein are useful for holding biological moieties in place in an intra- or extra-vascular implant in a patient to allow the biological moiety to provide biological therapy to a patient. Although the examples provided in the are focused on vascular implants and blood vessels, it is within the scope of this disclosure to deliver implantable devices intra-luminally, extra-luminally, or surgically to any conduit within any tissue bed within a body. U.S. Pat. No. 9,642,693 to Cully et al.; U.S. Pat. No. 8,197,529 to Cully, et. al.; U.S. Pat. No. 10,111,741 to Michalak; U.S. Pat. No. 10,779,980 to Sharma, et. al.; U.S. Pat. No. 9,717,584 to Cully, et. al; U.S. Pat. No. 6,673,102 to Vonesh, et al.; U.S. Pat. No. 7,914,568 to Cully et. al.; U.S. Pat. No. 6,673,102 to Vonesh, et al.; and U.S. Pat. No. 6,352,561 to Leopold, et al. exemplify device deployment in various tissue beds, both via intra-luminal placement and extra-luminal placement of the encapsulation device. Minimally invasive procedures utilizing conduits of vasculature and the gastrointestinal tract have been previously disclosed, such as, for example, in U.S. Pat. No. 7,914,568 to Cully et al.; U.S. Pat. No. 6,673,102 to Vonesh, et al.; and U.S. Pat. No. 6,352,561 to Leopold, et al.

In at least one embodiment, the encapsulation device is placed using a minimally invasive method. One non-limiting method utilizes a catheter and the method of placing the encapsulation device includes accessing a body conduit, tracking a catheter to a target location point on the body conduit, and deploying the encapsulation device within the body conduit. The encapsulation device includes at least one reservoir space that contains a biological moiety. The encapsulation device also includes a first cellular open layer on a first side of the at least one reservoir space. The cellular open layer faces the inner surface of the body conduit. A cell retentive layer is positioned on a second side of the reservoir space. In some embodiments, the at least one reservoir space is positioned within a containment layer. The reservoir space has at least one structural element and a separation distance, and the separation distance is maintained both under external compressive forces and under internal expansive forces. It is to be appreciated that the encapsulation device is configured to fit within the body conduit at the target location point. In some embodiments, the encapsulation device may include a reinforcing layer positioned between an outer layer and an inner layer and adjacent to the containment layer or a reinforcing layer positioned exteriorly to the outer layer.

In another embodiment, the method of placing the encapsulation device includes accessing a body conduit, tracking a catheter to a target location point, and deploying the encapsulation device at the target location point. The encapsulation device includes at least one reservoir space containing a biological moiety and that has at least one structural element located therein. A separation distance is maintained during deployment. The encapsulation device has a first cellular open layer on a first side of the at least one reservoir space and which faces the inner surface of the body conduit. A second cellular open layer may be positioned on a second side of the at least one reservoir space. In some embodiments, a cell retentive layer is positioned on the second side of the at least one reservoir space. A cell retentive layer may be positioned on at least one of the first cellular open layer and the second cellular open layers. In some embodiments, the separation distance is maintained both under external compressive forces and under internal expansive forces. It is to be appreciated that the encapsulation device is configured to fit within the body conduit at the target location point. In some embodiments, the encapsulation device may include a reinforcing layer positioned between an outer layer and an inner layer and adjacent to the containment layer. In other embodiments, the a reinforcing layer may be positioned externally to the outermost layer of the encapsulation device. In addition, the encapsulation device may contain a containment layer in which the at least one reservoir space and the at least one structural element is positioned.

In one embodiment of placing an extra-luminal device, the method includes accessing a first body conduit, inserting a guidewire into the first body conduit, using an accessory tool to exit the first body conduit at a target exit point, tracking the guidewire to a target entry point on a second body conduit, using the accessory tool to enter the second body conduit, and deploying an encapsulation device at the target entry point on the second body conduit. The encapsulation device includes at least one reservoir space containing therein a biological moiety, the reservoir space having a cell retentive layer thereon. In such an embodiment, the encapsulation device forms a third conduit connecting the first body conduit and the second body conduit. A cell retentive layer forms an inner layer of the third body conduit. The encapsulation device may also include a containment layer that includes the at least one reservoir space and contains structural elements that maintain a separation distance under external compressive forces and under internal expansive forces. In some embodiments, the encapsulation device includes a second cell retentive layer on a side of the at least one reservoir space opposing the first cell retentive layer.

In another embodiment, an encapsulation device may be utilized to bypass a desired bypassed region (e.g., an occlusion) in a body conduit. In at least one embodiment, a method includes accessing a first body conduit at a first target entry point, inserting a guidewire into the body conduit at the first target entry point, tracking a catheter over the guidewire to a target exit point on the first body conduit located before the desired bypassed region, using an accessory tool to exit the body conduit at a first target exit point, tracing the guidewire to a target second entry point on the body conduit at a location after the desired bypassed region, using the accessory tool to enter the body conduit at the second target entry point, and deploying an encapsulation device at the second target entry point such that the encapsulation device connects the first target exit point and the second target entry point. The encapsulation device includes at least one reservoir space containing a biological moiety therein and a cellular open layer that, once deployed, becomes the outer surface of the deployed device and is in contact with the inner surface of the body conduit to allow vascularization into the cellular open layer. In some embodiments, a cell retentive layer is positioned on at least one side of the at least one reservoir space.

Deployment of an extra-luminal implant may be performed using an open surgical procedure or a minimally invasive procedure such as, but not limited to, trans jugular intrahepatic portosystemic shunt (TIPS), which is described in U.S. Pat. No. 6,673,102 to Vonesh, et al. The cellular open layer allows ingrowth of vascular cells from, for example, a blood vessel. The encapsulation devices described herein may also be utilized in an open surgical procedure. In one non-limiting embodiment, a method of placing the encapsulation device includes accessing a first body conduit at a first target entry point, inserting a guidewire into the first body conduit, tracking a catheter over the guidewire to a target exit point from the first body conduit, using an accessory tool to exit the first body conduit, tracing the guidewire to a second target entry point on a second body conduit, using the accessory tool to access the second body conduit, and deploying the encapsulation device at the second target entry point such that the encapsulation device connects the first body conduit and the second body conduit. The encapsulation device may include at least one reservoir space that contains therein a biological moiety where the reservoir space has a cell retentive layer adjacent thereto. The at least one reservoir space is formed between structural elements. Both the at least one reservoir space and the structural element(s) are positioned within a containment layer. A cell retentive layer of the encapsulation device forms an inner layer of the third body conduit. In another embodiment, a cell retentive layer is positioned on the first side of the containment layer and a cellular open layer is positioned on the cell retentive layer and is adjacent to an inner surface of the body conduit.

In another embodiment of utilizing an open surgical procedure, a target location point on a body conduit is surgically accessed, a slit is formed on the body conduit to access the inside of the body conduit, and an encapsulation device is placed within the body conduit. The encapsulation device includes at least one reservoir space therein for containing a biological moiety. The at least one reservoir space has a cellular open layer positioned on an inner surface of the body conduit.

In yet another embodiment of an open surgical procedure, a method includes surgically accessing a first target location point in a first body conduit, connecting a first end of an encapsulation device to the first target location point of the first body conduit, and connecting a second end of the encapsulation device to a second target location point on a second body conduit where the cell retentive layer forms an inner surface of the third conduit. The encapsulation device includes a cell retentive layer and at least one reservoir space containing therein at least one biological moiety. The encapsulation device forms a third body conduit connecting the first body conduit and the second body conduit.

In a further embodiment, a method of placing the encapsulation device includes surgically accessing a body conduit and at a target location point such that the encapsulation device substantially surrounds at least a portion of the body conduit. The encapsulation device includes at least one reservoir space therein containing a biological moiety. In addition, the at least one reservoir space a cellular open layer is position on a side of the at least one reservoir space and is and is adjacent to the outside of the body conduit.

Another method of placing an intra-luminal device includes deploying an encapsulation device of any one of the embodiments described herein at a target location in a body conduit such that the outer layer is facing a wall of the body conduit, where the outer layer is a cellular open layer. The method may also include accessing the body conduit at a target location point a tracking a catheter over a guidewire to the target location point. In at least one embodiment, the device is constrained within the catheter. The encapsulation device may be cystoscopically deployed, laparoscopically deployed, or bronchoscopically deployed. It is to be appreciated that the encapsulation device is configured to fit the body conduit.

Another method of placing an intra-luminal device includes deploying an encapsulation device of any one of the embodiments described herein at a target location point in a body conduit such that the outer layer is facing a wall of the body conduit where the outer layer is a cellular open layer. The method may also include accessing the body conduit at a target location point a tracking a catheter over a guidewire to the target location point. In at least one embodiment, the device is constrained within the catheter. The encapsulation device may be cystoscopically deployed, laparoscopically deployed, or bronchoscopically deployed. It is to be appreciated that the encapsulation device is configured to fit the body conduit.

A method of placing an extra-luminal device includes accessing a first body conduit at a first target location point, creating a target exit point in the first body conduit at a second target location point, tracking a catheter over a guidewire to a target exit point on the first body conduit, exiting the first body conduit at the target exit point, tracking the guidewire to a target entry point on a second body conduit, deploying an encapsulation device of any one of the embodiments described herein at the target entry point on the second body conduit such that the encapsulation device interconnects the first body conduit and the second body conduit.

Another method of placing an extra-luminal device includes accessing a body conduit at a first target entry point at a first target location point on a body conduit, tracking a guidewire over a catheter to a first target exit point at a second target location point on the body conduit, exiting the body conduit at the first target exit point, tracking the catheter over the guidewire to a second target entry point at a third target location point on the body conduit, entering the body conduit at the second target entry point, and deploying an encapsulation device of any one of the embodiments described herein. The method may also include creating the second target entry point at the third target location point on the body conduit.

In another method for placing an extra-luminal device includes accessing a body conduit at a first target entry point at a first location on the first body conduit, tracking a guidewire over a catheter to a first target exit point at a second location on the first body conduit, exiting the first body conduit at the first target exit point, tracking the catheter over the guidewire to a second target entry point at a third location on a second body conduit, entering the second body conduit at the second target entry point, and deploying an encapsulation device of any one of the embodiments described herein.

A method for surgically placing an encapsulation device includes surgically accessing a target location point on a first body conduit at a first target entry point, resecting a portion of the body conduit at the target location point, replacing the resected portion of the body conduit at the target location point with an encapsulation device described herein via end-to-end anastomosis where the encapsulation device contains therein a biological moiety.

Another method for placing an encapsulation device includes surgically accessing a target location point on a body conduit, forming a slit in the body conduit at the target location point to access the body conduit, and inserting an encapsulation device described herein into the body conduit at the target location point.

In a further method for placing an encapsulation device includes surgically accessing a first target location point on a first body conduit, connecting a first end of an encapsulation device of any one of the embodiments described herein to the first target location point of the first body conduit, and connecting a second end of the encapsulation device to a second target location point on a second body conduit.

It is to be appreciated that the encapsulation devices described with respect to the methods of placing the encapsulation device includes any of the embodiments described herein and may contain any or all of the components forming the encapsulation devices described herein.

### Example

A mandrel including a proximal portion and a distal portion and a shaft length was obtained. A first release film including Kapton was wrapped onto the proximal portion of the mandrel. Next, a first ePTFE membrane that included a cellular open layer was wrapped over the first release film. A second ePTFE membrane that included a reinforcing layer was wrapped over the first ePTFE membrane continuing on to the distal portion of the mandrel. A third ePTFE membrane including a cell retentive membrane was then wrapped over the second ePTFE membrane.

A cylindrical laser cut stent frame was obtained that included superelastic Nitinol of a length shorter than the mandrel length. The stent frame was slid over the third ePTFE membrane such that the stent frame was oriented over the proximal portion of the mandrel. A second release film that included Kapton was then wrapped over the cylindrical stent frame.

The mandrel comprising the films, membranes, and stent frame, was heated at 350° C. for 15 min, to bond the ePTFE membranes to another. After heating, the mandrel was removed from the oven, the second release film was removed from the mandrel, and a film comprising fluorinated ethylene-propylene (FEP) was wrapped over the distal end of the stent frame.

A fourth ePTFE membrane that included structural elements was wrapped over the stent frame portion of the mandrel. The distal portion of the wrapped membrane comprising the second ePTFE membrane was inverted and slid axially up to the proximal portion of the mandrel, such that the stent frame was covered by the inverted second ePTFE membrane.

An FEP filling tube was inserted between the wrapped second ePTFE membrane and the inverted second ePTFE membrane at the proximal portion of the mandrel. The FEP filling tube was bonded to the ePTFE membrane using a soldering iron at 320° C. The entire assembly was removed from the mandrel.

The resulting encapsulation device has a structure similar to that depicted in FIG. 1A, but with the inclusion of the Nitinol stent frame positioned between the layer containing the structural elements and the second porous layer, and a filling tube positioned between the structural elements in a reservoir space.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An implantable encapsulation device comprising:
an inner layer;
an outer layer; and
a containment layer positioned between the inner layer and the outer layer and including structural elements disposed therein to maintain a separation distance between the inner layer and the outer layer, the structural elements defining a plurality of reservoir spaces for the placement of at least one biological moiety therein,
wherein the structural elements maintain the separation distance both under external compressive forces and under internal expansive forces,
wherein at least one of the inner layer and the outer layer is a composite layer comprising a cellular open layer and a cell retentive layer, and
wherein the encapsulation device has a substantially tubular configuration and is configurable from a first tubular configuration having a first diameter to a second tubular configuration having a second diameter.

2. The encapsulation device of claim 1, comprising a filling tube positioned between the structural elements and into at least one of the reservoir spaces for placement of the biological moiety in the reservoir spaces.

3. The encapsulation device of claim 1, comprising a reinforcing layer positioned between the inner layer and the containment layer.

4. The encapsulation device of claim 1, comprising a reinforcing layer positioned between the outer layer and the containment layer.

5. The encapsulation device of claim 1, comprising a reinforcing layer positioned externally to the outer layer.

6. The encapsulation device of claim 3, wherein the reinforcing layer comprises a shape memory material.

7. The encapsulation device of claim 1, wherein the inner layer is a composite layer comprising a cellular open layer and a first cell retentive layer, and
wherein the outer layer is a second cell retentive layer.

8. The encapsulation device of claim 1, wherein the outer layer is a composite layer comprising a cellular open layer and a first cell retentive layer, and
wherein the inner layer is a second cell retentive layer.

9. The encapsulation device of claim 1, wherein the inner layer is a first composite layer comprising a first cellular open layer and a first cell retentive layer, and
wherein the outer layer is a second composite layer comprising a second cellular open layer and a second cell retentive layer.

10. The encapsulation layer of claim 1, wherein the structural elements comprise a shape memory material.

11. The encapsulation device of claim 1, wherein the at least one biological moiety is selected from cells, viruses, viral vectors, bacteria, proteins, antibodies, genes, DNA, RNA and combinations thereof.

12. The encapsulation device of claim 11, wherein the cells are selected from prokaryotic cells, eukaryotic cells, mammalian cells, non-mammalian cells, stem cells and combinations thereof.

13. The encapsulation device of claim 1, wherein at least one of the inner layer the outer layer is a cellular open layer, and wherein the at least one biological moiety is microencapsulated.

14. The encapsulation device of claim 1, wherein the structural elements are adhered to at least one of the first layer and the second layer.

15. The encapsulation device of claim 1, wherein the inner layer is a first composite layer comprising a first cellular open layer and the outer layer is a second composite layer comprising a second cellular open layer,
wherein the structural elements are adhered to the first and second cellular open layers of the first and second composite layers, and
wherein the structural elements do not penetrate into pores of the first cellular open layer or the second cellular open layer.

16. The encapsulation device of claim 1, wherein the at least two reservoir spaces that are fluidly interconnected.

17. The encapsulation device of claim 1, wherein the at least two reservoir spaces are discrete.

18. An encapsulation device comprising:
a first composite layer including a first cellular open layer and a first cell retentive layer;
a second composite layer including a second cellular open layer and a second cell retentive layer;
a containment layer positioned between the first composite layer and the second composite layer, the containment layer including structural elements disposed therein to maintain a separation distance between the first composite layer and the second composite layer, the structural elements defining a plurality of reservoir spaces for the placement of at least one biological moiety therein, and
at least one configuration element comprising a shape memory material,
wherein the encapsulation device is configurable between a first geometric configuration and a second geometric configuration.

19. The encapsulation device of claim 18, wherein the at least one configuration element is positioned between the first cellular open layer and the first cell retentive layer.

20. The encapsulation device of claim 18, wherein the at least one configuration element is positioned between the second cellular open layer and the second cell retentive layer.

21. The encapsulation device of claim 18, wherein the at least one first configuration element is positioned between the first cellular open layer and the first cell retentive layer and at least one second configuration element is positioned between the second cellular open layer and the second cell retentive layer.

22. The encapsulation device of claim 18, wherein the at least one configuration element is exteriorly positioned on the first cellular open layer, the first cellular open layer forming an exterior surface of the encapsulation device.

23. The encapsulation device of claim 18, wherein the at least one configuration element is exteriorly positioned on the second cellular open layer, the second cellular open layer forming an exterior surface of the encapsulation device.

24. The encapsulation device of claim 18, wherein at least one configuration element replaces the structural elements and is positioned between the first composite layer and the second composite layer.

25. The encapsulation device of claim 18, wherein the biological moiety is selected from cells, viruses, viral vectors, bacteria, proteins, antibodies, genes, DNA, RNA and combinations thereof.

26. The encapsulation device of claim 25, wherein the cells are selected from prokaryotic cells, eukaryotic cells, mammalian cells, non-mammalian cells, stem cells and combinations thereof.

27. The encapsulation device of claim 18, wherein the structural elements are adhered to the first cellular open layer of the first composite layer and the second cellular open layer of the second composite layer, and
wherein the structural elements do not penetrate into pores of the first cellular open layer or the second cellular open layer.

28. The encapsulation device of claim 18, wherein the plurality of reservoir spaces in the cell containment layer are interconnected.

29. The encapsulation device of claim 18, wherein the plurality of reservoir spaces in the cell containment layer are discrete.

30. The encapsulation device of claim 18, wherein the structural elements maintain the separation distance both under external compressive forces and under internal expansive forces.

31.-83. (canceled)
